(19) United States
(12) Patent Application Publication
(10) Pub. No.: US 2008/0025916 A1
(43) Pub. Date: Jan. 31, 2008

(54) Tailored Treatment Suitable for Different Forms of Mastocytosis

(75) Inventors: Jean-Pierre Kinet, Lexington, MA (US); Alain Moussy, Paris (FR)

Correspondence Address: FOLEY AND LARDNER LLP;SUITE 500, 3000 K STREET NW, WASHINGTON, DC, 20007, US

(73) Assignee: AB Science

(21) Appl. No.: 10/547,074

(22) PCT Filed: Feb. 27, 2004
(86) PCT No.: PCT/IB04/00907

§ 371 Date: Aug. 26, 2005

Related U.S. Application Data
(60) Provisional application No. 60/449,861, filed on Feb. 27, 2003.

(51) Int. Cl. A61K 31/7076; A61K 31/4439; A61K 49/00; A61P 35/00; A61K 31/506

(52) U.S. Cl. 424/9.1; 514/275; 514/342; 514/46

(57) ABSTRACT

The present invention relates to a method for a tailored treatment of mastocytosis comprising a) assessing whether or not a c-kit mutation at position 816 is detected in a sample of a patient and b) administering a specific 816 mutant c-kit inhibitor in case a mutation is detected in step a) or an inhibitor displaying efficacy on c-kit wild in case no mutation is detected in step a). The invention is more particularly suited 10 for treating category II, III and IV mastocytosis.

Tailored treatment suitable for different forms of mastocytosis The present invention relates to a method for a tailored treatment of mastocytosis comprising a) assessing whether or not a c-kit mutation at position 816 is detected in a sample of a patient and b) administering a specific 816 mutant c-kit inhibitor in case a mutation is detected in step a) or an inhibitor displaying efficacy on c-kit wild in case no mutation is detected in step a). The invention is more particularly suited for treating category II, III and IV mastocytosis.

Mastocytosis are a very heterogeneous group of disorders characterized by an abnormal accumulation of mast cells in different tissues, mainly in the skin and the bone marrow, but also in spleen, liver, lymph nodes, and the gastrointestinal tract, depending on the nature of the disease. They can affect humans of either sex at any age. Neoplasms of mast cells (MC) can be acute or chronic. Acute mast cell neoplasms are designated as MC leukemia. Chronic mast cell neoplasms may be localized or generalized. Cutaneous mastocytosis is the commonest localized neoplasm and is often found in children in which it often remits and never relapses. Mastocytosis are usually acquired diseases, but some rare familial cases have been described.

With regard to the extreme heterogeneity of mast cell neoplasms, it is important to classify these diseases. One of the most used classification is the one by Metcalfe (Metcalfe, J Invest Dermatol. 96: 2S-4S, 1991) that distinguishes four categories of mastocytosis:

The category I is composed by two sub-categories (IA and IB). Category IA is made by diseases in which mast cell infiltration is strictly localized to the skin. This category represents the most frequent form of the disease and includes : i) urticaria pigmentosa, the most common form of cutaneous mastocytosis, particularly encountered in children, ii) diffuse cutaneous mastocytosis, iii) solitary mastocytoma and iv) some rare subtypes like bullous, erythrodermic and teleangiectatic mastocytosis. These forms are characterized by their excellent prognosis with spontaneous remissions in children and a very indolent course in adults. Long term survival of this form of disease is generally comparable to that of the normal population and the translation into another form of mastocytosis is rare. Category IB is represented by indolent systemic disease (SM) with or without cutaneous involvement. These forms are much more usual in adults than in children. The course of the disease is often indolent, but sometimes signs of aggressive or malignant mastocytosis can occur, leading to progressive impaired organ function.

The category II includes mastocytosis with an associated hematological disorder, such as a myeloproliferative or myelodysplastic syndrome, or acute leukemia. These malignant mastocytosis does not usually involve the skin. The progression of the disease depends generally on the type of associated hematological disorder that conditiones the prognosis.

The category III is represented by aggressive systemic mastocytosis in which massive infiltration of multiple organs by abnormal mast cells is common. In patients who pursue this kind of aggressive clinical course, peripheral blood features suggestive of a myeloproliferative disorder are more prominent. The progression of the disease can be very rapid, similar to acute leukemia, or some patients can show a longer survival time.

Finally, the category IV of mastocytosis includes the mast cell leukemia, characterized by the presence of circulating mast cells and mast cell progenitors representing more than 10% of the white blood cells. This entity represents probably the rarest type of leukemia in humans, and has a very poor prognosis, similar to the rapidly progressing variant of malignant mastocytosis. Mast cell leukemia can occur either de novo or as the terminal phase of urticaria pigmentosa or systemic mastocytosis.

Since categories II and III do not differ prognostically, the classification of Metcalfe can be further simplified as shown in Table I, according to the recommendations of Horny et al (Horny et al, Am J Surg Pathol. 22: 1132-40, 1998).

TABLE I

| Localized (category I) | Generalized (categories II, III, IV) |
|---|---|
| Cutaneous mastocytosis | Systemic mastocytosis (with or without |
| | cutaneous involvement) |
| Solitary mastocytoma | Indolent |
| Urticaria pigmentosa | Aggressive |
| | Mast cell leukemia |

Mast cells are characterized by their heterogeneity, not only regarding tissue location and structure but also at the functional and histochemical levels (Aldenborg and Enerback., Histochem. J. 26: 587-96, 1994; Bradding et al. J Immunol. 155: 297-307, 1995; Irani et al, J Immunol. 147: 247-53, 1991; Miller et al, Curr Opin Immunol. 1: 637-42, 1989 and Welle et al, J Leukoc Biol. 61: 233-45, 1997). Differentiation, survival and proliferation of MC depend greatly on SCF (Torrey et al, 1990). The receptor for SCF is c-kit, encoded by the protooncogene c-kit; it belongs to type III receptor tyrosine kinase subfamily (Baghestanian et al, 1996). Numerous studies have been performed regarding the neoplastic mechanism of mastocytosis, searching for genetic abnormalities of c-kit (mutation, deletion). The existence of such abnormalities was suggested because they were previously found in rodent or human leukemic MC lines. In human mastocytosis, mutations of c-kit have been described in vivo in various forms of mastocytosis (cutaneous mastocytosis, systemic indolent or systemic aggressive mastocytosis). Among the mutations found, the most common is the activating mutation Asp to Val at codon 816. In addition, one report has described a mutation in the juxtamembrane domain of c-kit (Val to Gly at codon 560) in human mastocytosis (Valent et al, 1994). Furthermore, Longley et al (Pauls et al, 1999) have showed that the point mutations in position 816.

As of today, the clinical suspicion of mastocytosis is confirmed by histologic examination, especially of skin and bone marrow. Stains such as tuoluidine blue can be used to identify mast cells by staining their metachromatic granules. Also, the chloroacetate-esterase reaction can complete staining. In addition, immunocytochemistry for tryptase is useful to confirm the nature of the cellular infiltrate. Finally, the diagnostic can be helped by the use of the immunophenotyping of MC in bone marrow aspirate. Indeed, normal as well as mastocytosis-related mast cells strongly express CD117 antigen (Arber et al, Hum Pathol. 29: 498-504, 1998; Escribano et al, Cytometry. 30: 98-102, 1997), and some antigens not found on normal MC can be aberrantly expressed by neoplastic mast cells, such a CD2, CD25 and CD35 (Escribano et al Blood. 91: 2731-6, 1998, Ormerod et al, British Journal of Dermatology. 122: 737-44, 1990). Other findings have shown that the CD69 activation antigen is over-expressed on MC from patients with systemic mastocytosis, as compared to normal mast cells (Diaz-Agustin et al, Br J Haematol. 106: 400-5, 1999).

Biochemical determination of mast cell mediators can also help to diagnosis of mastocytosis: histamine level in blood and urine, metabolites of prostaglandin D2 and of histamine in the urine are increased in most cases of SM, as well as the level of tryptase in blood (Hogan and Schwartz, Methods 13: 43-52, 1997˜Van Gysel et_al, J Am Acad Dermatol. 35: 556-8, 1996˜Morrow et al, J Invest Dermatol. 104: 937-40, 1995; Marone et al, Chem Immunol. 62: 1-21, 1995). However, with these tests, some false positive (allergy) or false negative (mastocytosis without mediator release) may exist.

A number of studies have been performed to elucidate whether mutations of c-kit are associated with different clinical forms of mast cell diseases. Indeed, mutations of c-kit have been described in vivo in various forms of mastocytosis (cutaneous mastocytosis, systemic indolent or systemic aggressive mastocytosis). Among the mutations found, the there are the Asp to Val at codon 816 and juxtamembrane mutation.

Asp816Val mutation occurs in an early progenitor cell, and not in mature mast cells since it is carried by myelomonocytic cells, T cells, and B cells in addition to MC. The same activating point mutations in codon 816 of the c-kit gene have been described not only in patients with isolated mastocytosis but also in mastocytosis with an associated Hematological disorder, such as a myeloproliferative or myelodysplastic syndrome, or acute leukemia (Boissan and Arock, Leukoc Biol. 67: 135-48,2000).

More recently, we have proposed to use standard molecular biology techniques based on PCR in our patent application WO 03/002114 for precisely determining the activating mutation in a given patient. Probes and primers have been designed so as to be specific to such mutations analysis. We also described methods for identifying non-toxic specific c-kit inhibitors that are either active on SCF activated c-kit or on constitutively activated c-kit, notably on the 816 mutant c-kit, in our application WO 03/003006.

We now have discovered that 60% of patients suspected to be afflicted with different forms of mastocytosis bear a mutation at the 816 position of c-kit. This observation has been possible by performing of a genotyping study on about 200 patients.

Unexpectedly, we also observed that diagnostic methods using bone marrow as sample are not accurate and predictable enough to classify patients falling either in i) the 816 bearing mutation category or ii) the non-816 bearing mutation category. On the contrary, methods based on skin sample have revealed accurate for that purpose.

Therefore, the invention provides a tailored treatment to patients belonging either to i) or ii) category allowing the administration of a relevant c-kit inhibitor. This is particularly useful since c-kit inhibitors may be active on 816-mutated c-kit or on c-kit wild or other forms of c-kit but not on both. The choice of the appropriate inhibitor is of great importance considering the inefficacy and the potential side effects of improperly administered c-kit inhibitors.

### DESCRIPTION

In a first embodiment, the invention contemplates a method for a tailored treatment of mastocytosis comprising a) assessing whether or not a c-kit mutation at position 816 is detected in a sample of a patient and b) administering a specific 816 mutant c-kit inhibitor in case a mutation is detected in step a) or an inhibitor displaying efficacy on c-kit wild and/or on juxtamembrane mutated c-kit in case no mutation is detected in step a).

In addition, the invention relates to a method as defined above for treating category I, II, III and IV mastocytosis in human and any symptom associated with category I, II, III and IV mastocytosis. More specifically, the method according to the invention is useful for treating urticaria pigmentosa, diffuse cutaneous mastocytosis, solitary mastocytoma in human, as well as dog mastocytoma and some rare subtypes like bullous, erythrodermic and teleangiectatic mastocytosis, mastocytosis with an associated hematological disorder, such as a myeloproliferative or myelodysplastic syndrome, or acute leukemia, myeloproliferative disorder associated with mastocytosis, and mast cell leukemia.

Specific inhibitors of the 816 mutated c-kit or specific c-kit wild or juxtamembrane mutated c-kit inhibitors can be identified according to the method as described in WO 03/003006. Following this teaching, the man skilled in the art can routinely test compounds selected from bis monocyclic, bicyclic or heterocyclic aryl compounds (WO 92/20642), vinylene-azaindole derivatives (WO 94/14808) and 1-cycloproppyl-4-pyridyl-quinolones (U.S. Pat. No. 5,330,992), Styryl compounds (U.S. Pat. No. 5,217,999), styryl-substituted pyridyl compounds (U.S. Pat. No. 5,302,606), seleoindoles and selenides (WO 94/03427), tricyclic polyhydroxylic compounds (WO 92/21660) and benzylphosphonic acid compounds (WO 91/15495), pyrimidine derivatives (U.S. Pat. No. 5,521,184 and WO 99/03854), indolinone derivatives and pyrrol-substituted indolinones (U.S. Pat. No. 5,792,783, EP 934 931, U.S. Pat. No. 5,834,504, U.S. Pat. No. 5,883,116, U.S. Pat. No. 5,883,113, U.S. Pat. No. 5, 886,020, WO 96/40116 and WO 00/38519), as well as bis monocyclic, bicyclic aryl and heteroaryl compounds (EP 584 222, U.S. Pat. No. 5,656,643 and WO 92/20642), quinazoline derivatives (EP 602 851, EP 520 722, U.S. Pat. No. 3,772,295 and U.S. Pat. No. 4,343,940) and aryl and heteroaryl quinazoline (U.S. Pat. No. 5,721,237, U.S. Pat. No. 5,714,493, U.S. Pat. No. 5,710,158 and WO 95/15758).

In connection with the present invention, said c-kit inhibitor is a non-toxic, selective, potent and specific inhibitor of either the 816 mutant or c-kit wild or jutamembrane mutated form.

Such inhibitors can be selected from the group consisting of 2-(3-amino)arylamino-4-aryl-thiazoles, pyrimidine derivatives such as N-phenyl-2-pyrimidine-amine derivatives (U.S. Pat. No. 5,521,184 and WO 99/03854), indolinone derivatives and pyrrol-substituted indolinones (U.S. Pat. No. 5,792,783, EP 934 931, U.S. Pat. No. 5,834,504), U.S. Pat. No. 5,883,116, U.S. Pat. No. 5,883,113, U.S. Pat. No. 5,886,020, WO 96/40116 and WO 00/38519), as well as bis monocyclic, bicyclic aryl and heteroaryl compounds (EP 584 222, U.S. Pat. No. 5,656,643 and WO 92/20642), quinazoline derivatives (EP 602 851, EP 520 722, U.S. Pat. No. 3,772,295 and U.S. Pat. No. 4,343,940), 4-amino-substituted quinazolines (U.S. Pat. No. 3,470,182), 4-thienyl-2-(1H)-quinazolones, 6,7-dialkoxyquinazolines (U.S. Pat. No. 3,800,039), aryl and heteroaryl quinazoline (U.S. Pat. No. 5,721,237, U.S. Pat. No. 5,714,493, U.S. Pat. No. 5,710,158 and WO 95/15758), 4-anilinoquinazoline compounds (U.S. Pat. No. 4,464,375), and 4-thienyl-2-(1H)-quinazolones (U.S. Pat. No. 3,551,427).

In a particular embodiment, said specific inhibitor is an inhibitor of c-kit wild and is not potent inhibitor of c-kit 816 mutants. It can be selected from pyrimidine derivative, more particularly N-phenyl-2-pyrimidine-amine derivatives of formula I:

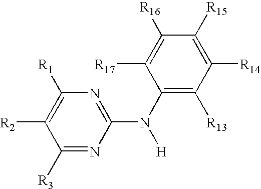

wherein the R1, R2, R3, R13 to R17 groups have the meanings depicted in EP 564 409 B1, incorporated herein in the description.

Preferably, the N-phenyl-2-pyrimidine-amine derivative is selected from the compounds corresponding to formula II:

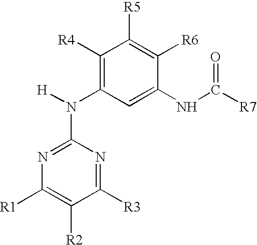

Wherein R1, R2 and R3 are independently chosen from H, F, Cl, Br, I, a C1-C5 alkyl or a cyclic or heterocyclic group, especially a pyridyl group;

R4, R5 and R6 are independently chosen from H, F, Cl, Br, I, a C1-C5 alkyl, especially a methyl group;

and R7 is a phenyl group bearing at least one substituent, which in turn possesses at least one basic site, such as an amino function.

Preferably, R7 is the following group:

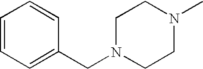

Among these compounds, the preferred are defined as follows:

R1 is a heterocyclic group, especially a pyridyl group,

R2 and R3 are H,

R4 is a C1-C3 alkyl, especially a methyl group,

R5 and R6 are H,

and R7 is a phenyl group bearing at least one substituent, which in turn possesses at least one basic site, such as an amino function, for example the group:

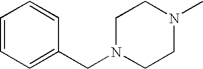

Therefore, in a preferred embodiment, the invention relates to a method as defined above comprising the administration of an effective amount of the compound known in the art as CGP57148B or STI571:

4-(4-méhylpipérazine-1-ylméthyl)-N-[4-méthyl-3-(4-pyridine-3-yl)pyrimidine-2 ylamino)phenyl]-benzamide corresponding to the following formula:

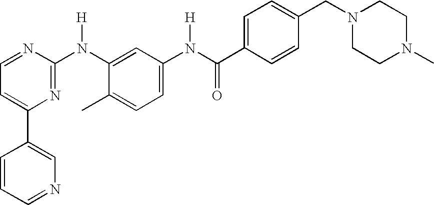

The preparation of this compound is described in example 21 of EP 564 409 and the β-form, which is particularly useful is described in WO 99/03854.

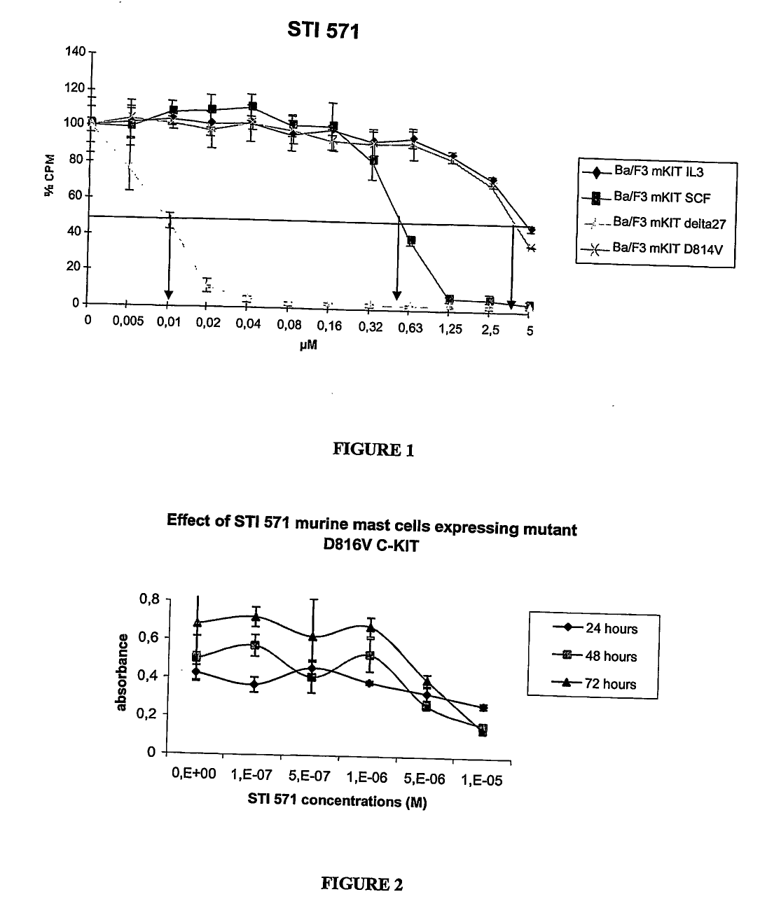

STI571 may be used in case step a) of the method according to the invention reveals no mutation at position 816. Indeed, this compound has been shown in WO 02/080925 to be very active on the juxtamembrane mutated c-kit and active on c-kit wild and we have demonstrated that it is not active on 816 mutated c-kit (see FIGS. 1 and 2).

In another embodiment, the invention is aimed at the use of compounds capable of specifically inhibiting 816 mutant c-kit to manufacture a medicament for treating the category of patients bearing the 816 mutation afflicted with Category II, III and IV mastocytosis, in particular mastocytosis with an associated hematological disorder, such as a myeloproliferative or myelodysplastic syndrome, or acute leukemia, myeloproliferative disorder associated with mastocytosis, and mast cell leukemia. By the expression “specifically inhibiting” it is meant to refer to compounds having an IC50 below 1 μM, preferably below 0.1 μM for ‘816’ mutated C-KIT and above 5 1 μM, preferably above 10 1 μM for C-KIT wild.

Such inhibitors can be selected from 2-(3-amino)arylamino-4-aryl-thiazoles such as those chosen from formula III, IV, V, VI, VII, VIIbis, and VIII for which the applicant filed PCT IB03/03685.

Alternatively, from these compounds of formula III, IV, V, VI, VII, VIIbis, and VIII, it is now possible to identify those which are specifically inhibiting C-KIT wild. Here, by the expression “specifically inhibiting” is meant to refer to compounds having an IC50 below 1 μM, preferably below 0.1 for C-KIT wild and above 5 μM, preferably above 10 μM for ‘816’ mutated C-KIT.

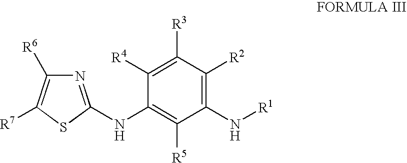

wherein R¹ is:
a) a linear or branched alkyl group containing from 1 to 10 carbon atoms optionally substituted with at least one heteroatom, notably a halogen selected from I, Cl, Br and F, or bearing a pendant basic nitrogen functionality;
b) an aryl or heteroaryl group optionally substituted by an alkyl or aryl group optionally substituted with an heteroatom, notably a halogen selected from I, Cl, Br and F or bearing a pendant basic nitrogen functionality;
c) a sulfonyl or a —SO2—R group wherein R is an alkyl, aryl or heteroaryl substituted with an heteroatom, notably a halogen selected from I, Cl, Br and F or bearing a pendant basic nitrogen functionality;
d) a —CO—NH—R, —CO—R, —CO—OR or a —CO—NRR′ group, wherein R and R′ are independently chosen from H or an aryl, heteroaryl, alkyl and cycloalkyl group optionally substituted with at least one heteroatom, notably a halogen selected from I, Cl, Br and F, or bearing a pendant basic nitrogen functionality;
R² is hydrogen, halogen or a linear or branched alkyl group containing from 1 to 10 carbon atoms, trifluoromethyl or alkoxy;
R³ is hydrogen, halogen or a linear or branched alkyl group containing from 1 to 10 carbon atoms, trifluoromethyl or alkoxy;
R⁴ is hydrogen, halogen or a linear or branched alkyl group containing from 1 to 10 carbon atoms, trifluoromethyl or alkoxy.
R⁵ is hydrogen, halogen or a linear or branched alkyl group containing from 1 to 10 carbon atoms, trifluoromethyl or alkoxy;
R⁶ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy,
iv) H, an halogen selected from I, F, Cl or Br; NH2, NO2 or SO2; and R⁷ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.
iv) H, an halogen selected from I, F, Cl or Br; NH2, NO2 or SO2.

Examples of compounds of formula III in which R1 corresponds to the definition given in a) (alkyl), b) (aryl) and d) (amide) are depicted below:
AB1

4-Diethylaminomethyl-N-[4-methyl-3-(4-pyridin-3-yl-thiazol-2-ylamino)-phenyl]-benzamide
AB2

N-[4-Methyl-3-(4-pyridin-3-yl-thiazol-2-ylamino)-phenyl]-4-morpholin-4-ylmethyl-benzamide
AB3

4-Dipropylaminomethyl-N-[4-methyl-3-(4-pyridin-3-yl-thiazol-2-ylamino)-phenyl]-benzamide
AB4

N-[4-Methyl-3-(4-pyridin-3-yl-thiazol-2-ylamino)-phenyl]-4-piperidin-1-ylmethyl-benzamide
AB5

3-Iodo-N-[4-methyl-3-(4-pyridin-3-yl-thiazol-2-ylamino)-phenyl]-benzamide
AB6

4-Hydroxymethyl-N-[4-methyl-3-(4-pyridin-3-yl-thiazol-2-ylamino)-phenyl]-benzamide
AB8

4-{[4-Methyl-3-(4-pyridin-3-yl-thiazol-2-ylamino)-phenylamino]-methyl}-benzoic acid methyl ester
AB11

3-Phenyl-propynoic acid [4-methyl-3-(4-pyridin-3-yl-thiazol-2-ylamino)-phenyl]-amide
AB14

4-Amino-N-[4-methyl-3-(4-pyridin-3-yl-thiazol-2-ylamino)-phenyl]-benzamide
AB16

2-Iodo-N-[4-methyl-3-(4-pyridin-3-yl-thiazol-2-ylamino)-phenyl]-benzamide
AB17

4-Iodo-N-[4-methyl-3-(4-pyridin-3-yl-thiazol-2-ylamino)-phenyl]-benzamide
AB18

4-(3-{4-[4-Methyl-3-(4-pyridin-3-yl-thiazol-2-ylamino)-phenylcarbamoyl]-phenyl}-ureido)-benzoic acid ethyl ester
AB20

N-[4-Methyl-3-(4-pyridin-3-yl-thiazol-2-ylamino)-phenyl]-4-[3-(4-trifluoromethyl-phenyl)-ureido]-benzamide
AB21

4-[3-(4-Bromo-phenyl)-ureido]-N-[4-methyl-3-(4-pyridin-3-yl-thiazol-2-ylamino)-phenyl]-benzamide
AB23

{4-[4-Methyl-3-(4-pyridin-3-yl-thiazol-2-ylamino)-phenylcarbamoyl]-benzyl}-carbamic acid tert-butyl ester
AB24

4-Hydroxy-N-[4-methyl-3-(4-pyridin-3-yl-thiazol-2-ylamino)-phenyl]-benzamide
AB26

4-[(Diisopropylamino)-methyl]-N-[4-methyl-3-(4-pyridin-3-yl-thiazol-2-ylamino)-phenyl]-benzamide
AB34

N-[4-Methyl-3-(4-pyridin-3-yl-thiazol-2-ylamino)-phenyl]-4-(3-thiophen-2-yl-ureido)-benzamide
AB35

4-[3-(3,5-Dimethyl-isoxazol-4-yl)-ureido]-N-[4-methyl-3-(4-pyridin-3-yl-thiazol-2-ylamino)-phenyl]-benzamide
AB37

4-[3-(4-Methoxy-phenyl)-ureido]-N-[4-methyl-3-(4-pyridin-3-yl-thiazol-2-ylamino)-phenyl]-benzamide
AB38

4-[3-(4-Difluoromethoxy-phenyl)-ureido]-N-[4-methyl-3-(4-pyridin-3-yl-thiazol-2-ylamino)-phenyl]-benzamide
AB39

Thiophene-2-sulfonic acid 4-[4-methyl-3-(4-pyridin-3-yl-thiazol-2-ylamino)-phenylcarbamoyl]-phenyl ester
AB40

4-Iodo-benzenesulfonic acid 4-[4-methyl-3-(4-pyridin-3-yl-thiazol-2-ylamino)-phenylcarbamoyl]-phenyl ester
AB41

N-[4-Methyl-3-(4-pyridin-3-yl-thiazol-2-ylamino)-phenyl]-4-pyrrolidin-1-ylmethyl-benzamide
AB42

3-Methyl-N-[4-methyl-3-(4-pyridin-3-yl-thiazol-2-ylamino)-phenyl]-benzamide
AB43

N-[4-Methyl-3-(4-pyridin-3-yl-thiazol-2-ylamino)-phenyl]-3-trifluoromethyl-benzamide
AB44
4-[3-(2,4-Dimethoxy-phenyl)-ureido]-N-[4-methyl-3-(4-pyridin-3-yl-thiazol-2-ylamino)-phenyl]-benzamide
AB45

N-[4-Methyl-3-(4-pyridin-3-yl-thiazol-2-ylamino)-phenyl]-4-[3-(4-trifluoromethyl-phenyl)-ureidomethyl]-benzamide
AB46

N-[4-Methyl-3-(4-pyridin-3-yl-thiazol-2-ylamino)-phenyl]-4-[3-(3,4,5-trimethoxy-phenyl)-ureido]-benzamide
AB48

4-[3-(2-Iodo-phenyl)-ureido]-N-[4-methyl-3-(4-pyridin-3-yl-thiazol-2-ylamino)-phenyl]-benzamide
AB49

4-[3-(4-Fluoro-phenyl)-ureido]-N-[4-methyl-3-(4-pyridin-3-yl-thiazol-2-ylamino)-phenyl]-benzamide
AB50

2-Fluoro-benzenesulfonic acid 4-[4-methyl-3-(4-pyridin-3-yl-thiazol-2-ylamino)-phenylcarbamoyl]-phenyl ester
AB51

3-Fluoro-benzenesulfonic acid 4-[4-methyl-3-(4-pyridin-3-yl-thiazol-2-ylamino)-phenylcarbamoyl]-phenyl ester

In another preferred embodiment, when R¹ has the meaning depicted in d) above, the invention is directed to compounds of the following formula IV:

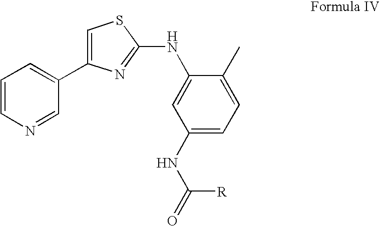

wherein R is H or an organic group that can be selected for example from a linear 25 or branched alkyl group containing from 1 to 10 carbon atoms optionally substituted with at least one heteroatom or bearing a pendant basic nitrogen functionality; a cycloalkyl, an aryl or heteroaryl group optionally substituted by an alkyl, a cycloalkyl, an aryl or heteroaryl group optionally substituted with an heteroatom, notably a halogen selected from I, Cl, Br and F or bearing a pendant basic nitrogen functionality.

Such compounds can be selected for example from:

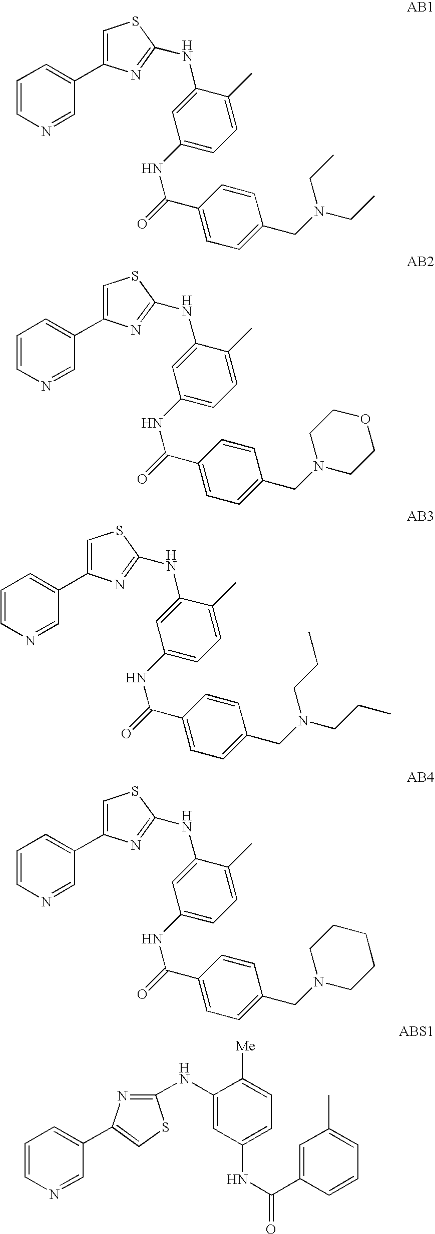

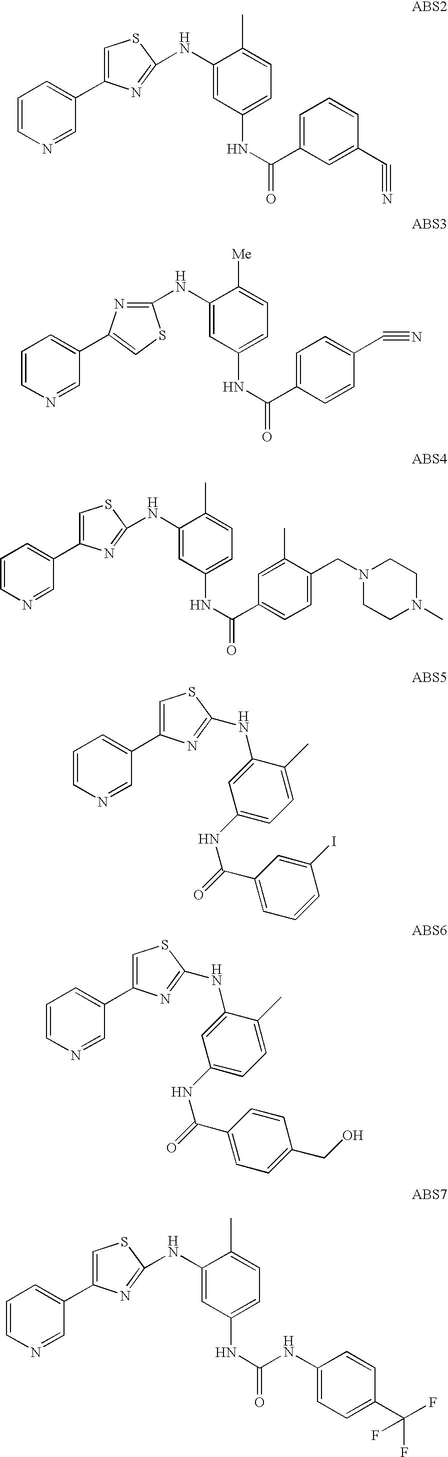

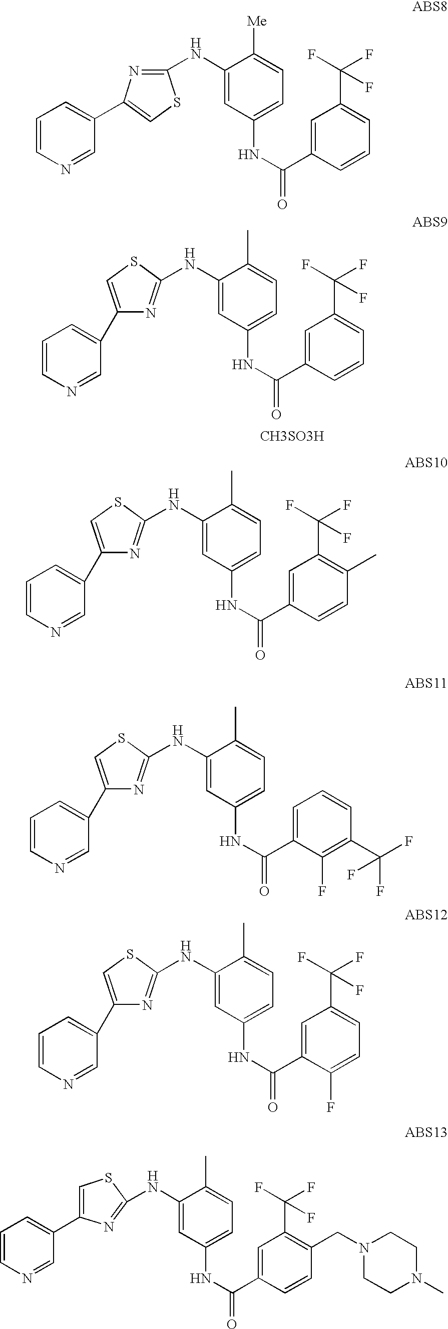

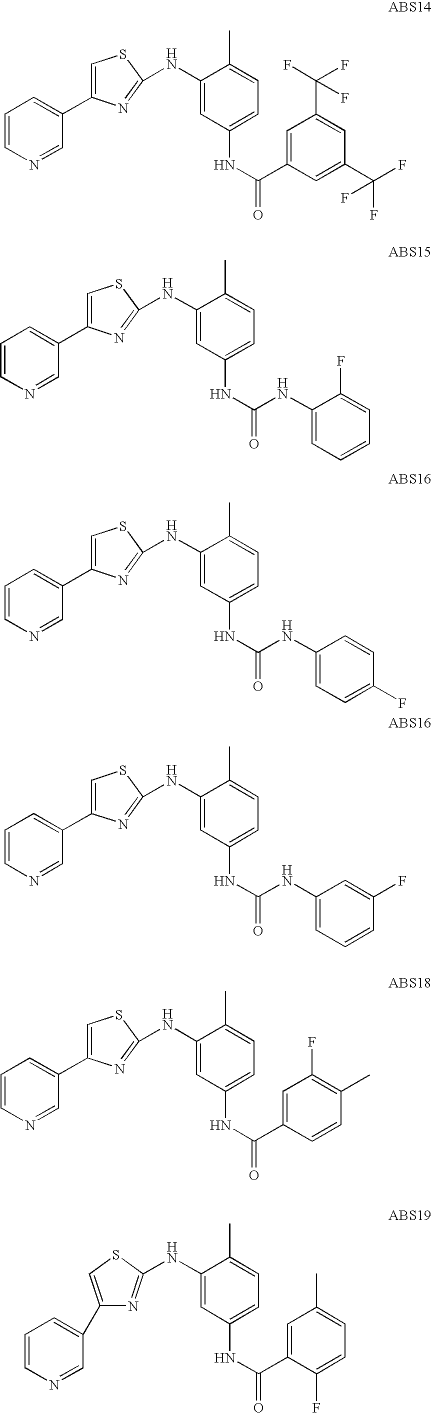

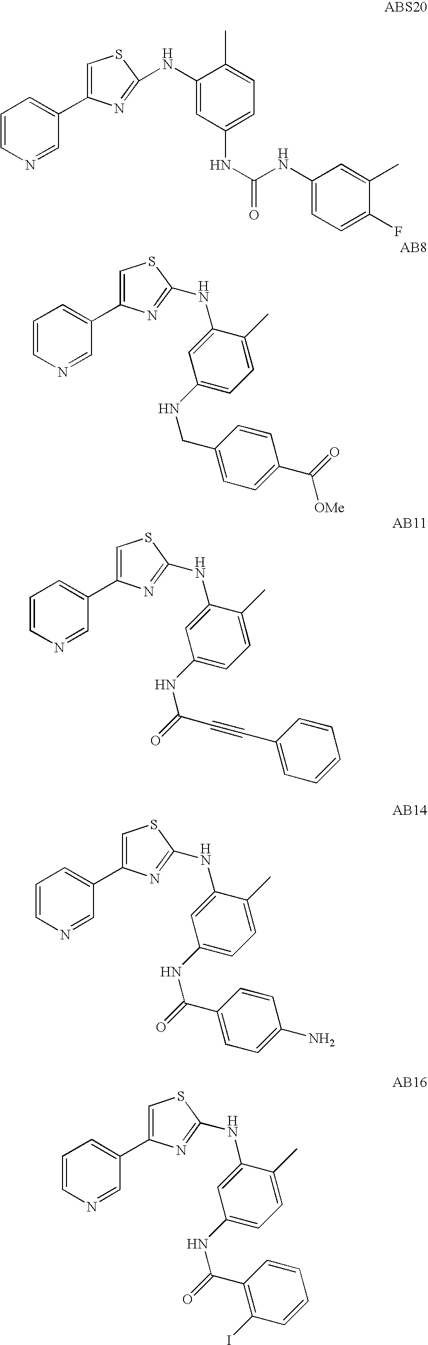

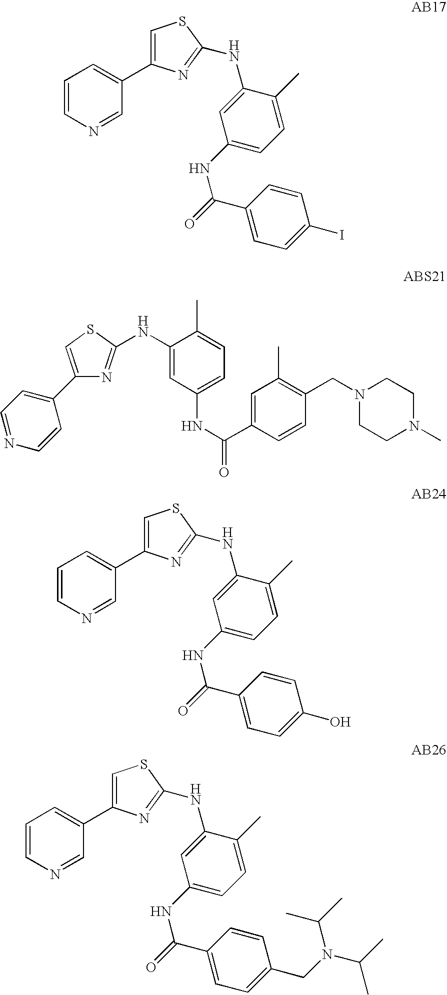

Among the particular compounds in which R1 has the meaning as depicted in d) above, the invention is directed to amide-aniline compounds of the following formula V:

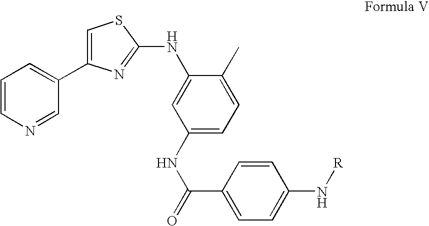

wherein R is H or an organic group that can be selected for example from a linear or branched alkyl group containing from 1 to 10 carbon atoms optionally substituted with at least one heteroatom or bearing a pendant basic nitrogen functionality; a cycloalkyl, an aryl or heteroaryl group optionally substituted with an heteroatom, notably a halogen selected from I, Cl, Br and F or bearing a pendant basic nitrogen functionality; or a a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;

a sulfonyl or a —SO2—R group wherein R is H, an alkyl, cycloalkyl, aryl or heteroaryl optionally substituted with an heteroatom, notably a halogen selected from I, Cl, Br and F or bearing a pendant basic nitrogen functionality; or a —CO—R or a —CO—NRR′ group, wherein R and R′ are independently chosen from H, an alkyl, a cycloalkyl, an aryl or heteroaryl group optionally substituted with at least one heteroatom, notably selected from I, Cl, Br and F, or bearing a pendant basic nitrogen functionality.

Examples of such compounds are as follows:

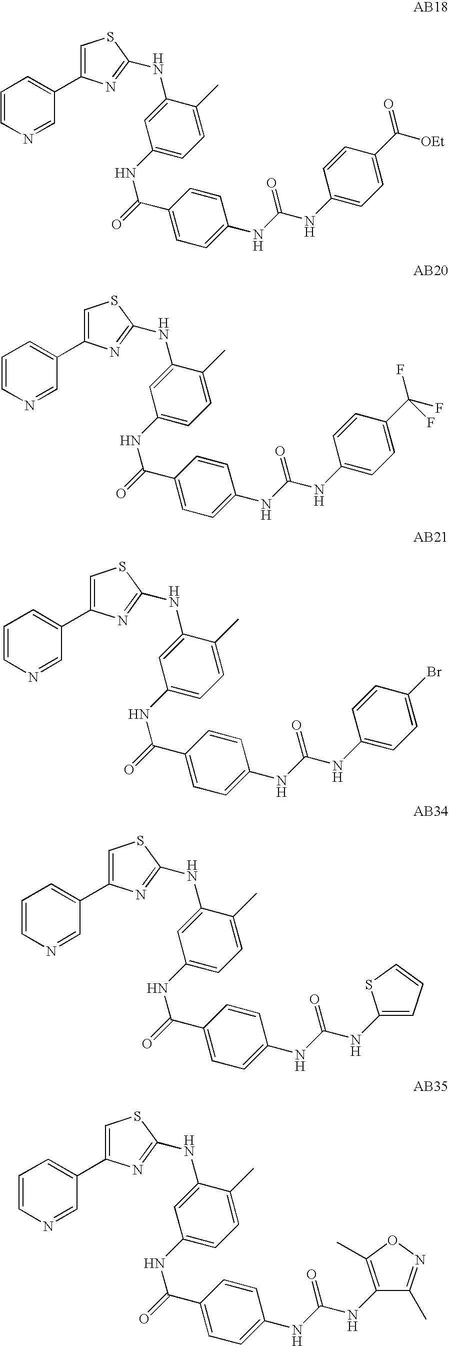

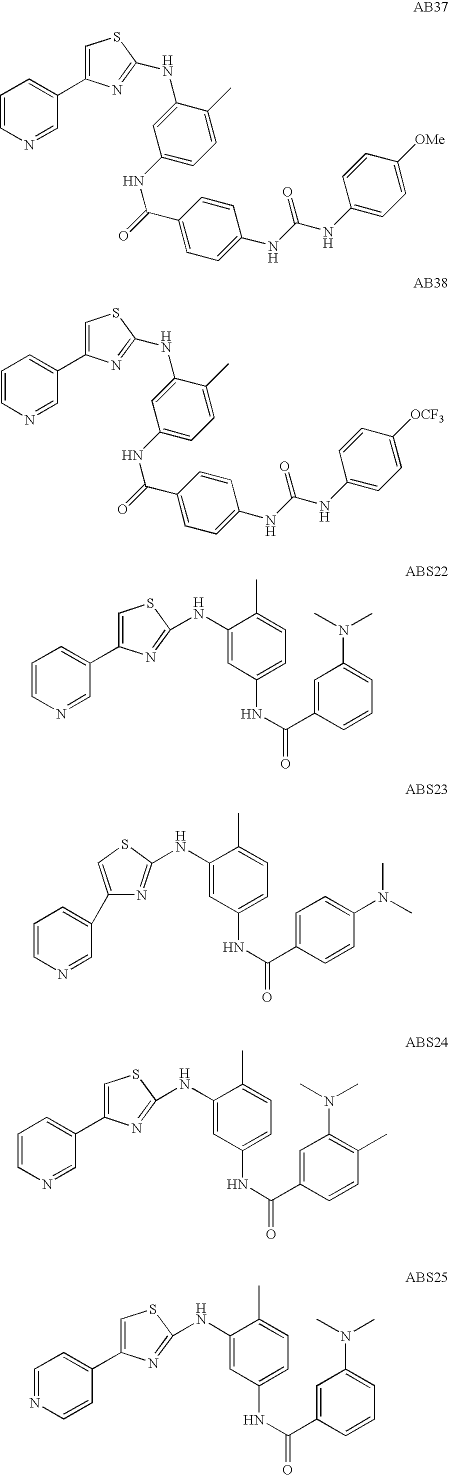

Among the particular compounds in which R1 has the meaning as depicted in d) above, the invention is directed to amide-benzylamine compounds of the following formula VI:

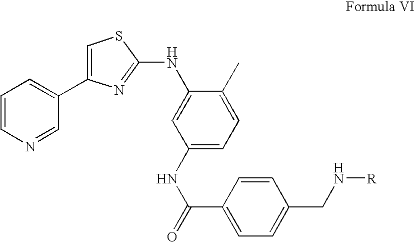

wherein R is H or an organic group that can be selected for example from a linear or branched alkyl group containing from 1 to 10 carbon atoms optionally substituted with at least one heteroatom, notably a halogen selected from I, Cl, Br and F, or bearing a pendant basic nitrogen functionality; a cycloalkyl, aryl or heteroaryl group optionally substituted with an heteroatom, notably a halogen selected from 1, Cl, Br and F or bearing a pendant basic nitrogen functionality; or an alky, cycloalkyl, aryl or heteroaryl group substituted by a alkyl, cycloalkyl, aryl or heteroaryl group optionally substituted with an heteroatom, notably a halogen selected from I, Cl, Br and F or bearing a pendant basic nitrogen functionality;

a sulfonyl or a —SO2—R group wherein R is H or an alkyl, cycloalkyl, aryl or heteroaryl group optionally substituted with an heteroatom, notably a halogen selected from I, Cl, Br and F or bearing a pendant basic nitrogen functionality; or a —CO—R or a —CO—NRR′ group, wherein R and R′ are independently chosen from H or an aryl heteroaryl, alkyl and cycloalkyl group optionally substituted with at least one heteroatom or bearing a pendant basic nitrogen functionality.

For example, this compound has the following formula:

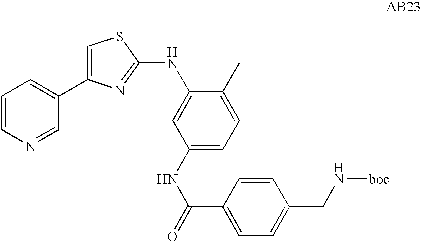

Among the particular compounds in which R1 has the meaning as depicted in d) above, the invention is directed to amide-phenol compounds of the following formula VI:

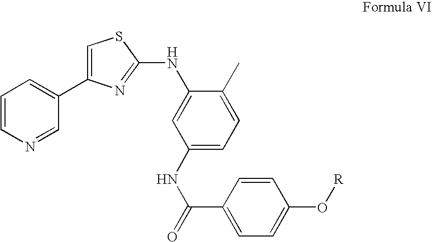

wherein R is H or an organic group that can be selected for example from a linear or branched alkyl group containing from 1 to 10 carbon atoms optionally substituted with at least one heteroatom, notably a halogen selected from L Cl, Br and F, or bearing a pendant basic nitrogen functionality;

a cycloalkyl, aryl or heteroaryl group optionally substituted with an heteroatom, notably a halogen selected from I, Cl, Br and F or bearing a pendant basic nitrogen functionality; or an alkyl, cycloalkyl, aryl or heteroaryl group substituted by a alkyl, cycloalkyl, aryl or heteroaryl group optionally substituted with an heteroatom, notably a halogen selected from I, Cl, Br and F or bearing a pendant basic nitrogen functionality;

a sulfonyl or a —SO2—R group wherein R is H or an alkyl, cycloalkyl, aryl or heteroaryl group optionally substituted with an heteroatom, notably a halogen selected from I, Cl, Br and F or bearing a pendant basic nitrogen functionality; or a —CO—R or a —CO—NRR′ group, wherein R and R′ are independently chosen from H or an aryl heteroaryl, alkyl and cycloalkyl group optionally substituted with at least one heteroatom or bearing a pendant basic nitrogen functionality.

Examples of such compounds are as follows:

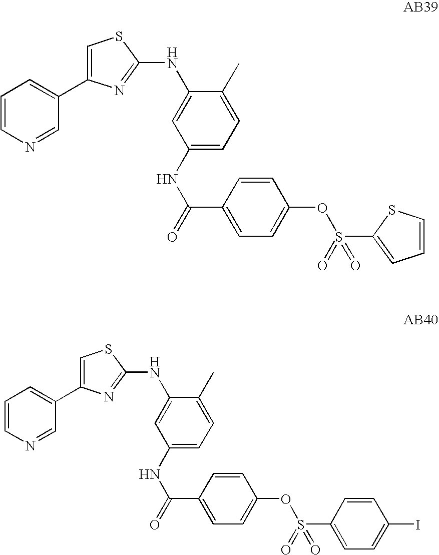

Among the particular compounds in which R1 has the meaning as depicted in d) above, the invention is directed to urea compounds of the following formula VII:

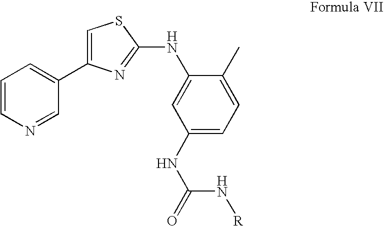

wherein R is H or an organic group that can be selected for example from a linear or branched alkyl group containing from 1 to 10 carbon atoms optionally substituted with at least one heteroatom (for example an halogen) or bearing a pendant basic nitrogen functionality; a cycloalkyl, an aryl or heteroaryl group optionally substituted with at least one heteroatom, notably a halogen selected from I, Cl, Br and F or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F or bearing a pendant basic nitrogen functionality.

Examples of such compounds are as follows:
AB7

1-(4-Methoxy-phenyl)-3-[4-methyl-3-(4-pyridin-3-yl-thiazol-2-ylamino)-phenyl]-urea
AB9

1-(4-Bromo-phenyl)-3-[4-methyl-3-(4-pylidin-3-yl-thiazol-2-ylamino)-phenyl]-urea
AB10

1-[4-Methyl-3-(4-pyridin-3-yl-thiazol-2-ylamino)-phenyl]-3-(4-trifluoromethyl-phenyl)-urea
AB12

1-(4-Fluoro-phenyl)-3-[4-methyl-3-(4-pyridin-3-yl-thiazol-2-ylamino)-phenyl]-urea
AB13

1-[4-Methyl-3-(4-pyridin-3-yl-thiazol-2-ylamino)-phenyl]-3-(3,4,5-trimethoxy-phenyl)-urea
AB15

4-{3-[4-Methyl-3-(4-pyridin-3-yl-thiazol-2-ylamino)-phenyl]-ureido}-benzoic acid ethyl ester
AB19

1-[4-Methyl-3-(4-pyridin-3-yl-thiazol-2-ylamino)-phenyl]-3-thiophen-2-yl-urea
AB22

1-Cyclohexyl-1-(N-Cyclohexyl-formamide)-3-[4-methyl-3-(4-pyridin-3-yl-thiazol-2-ylamino)-phenyl]-urea
AB25

1-(2,4-Dimethoxy-phenyl)-3-[4-methyl-3-(4-pyridin-3-yl-thiazol-2-ylamino)-phenyl]-urea
AB27

1-(2-Iodo-phenyl)-1-(N-(2-Iodo-phenyl)-formamide)-3-[4-methyl-3-(4-pyridin-3-yl-thiazol-2-ylamino)-phenyl]-urea
AB28

1-(3,5-Dimethyl-isoxazol-4-yl)-3-[4-methyl-3-(4-pyridin-3-yl-thiazol-2-ylamino)-phenyl]-urea
AB32

1-(2-Iodo-phenyl)-3-[4-methyl-3-(4-pyridin-3-yl-thiazol-2-ylamino)-phenyl]-urea
AB36

1-(4-Difluoromethoxy-phenyl)-3-[4-methyl-3-(4-pyridin-3-yl-thiazol-2-ylamino)-phenyl]-urea
AB47

1-(4-Dimethylamino-phenyl)-3-[4-methyl-3-(4-pyridin-3-yl-thiazol-2-ylamino) phenyl]-urea

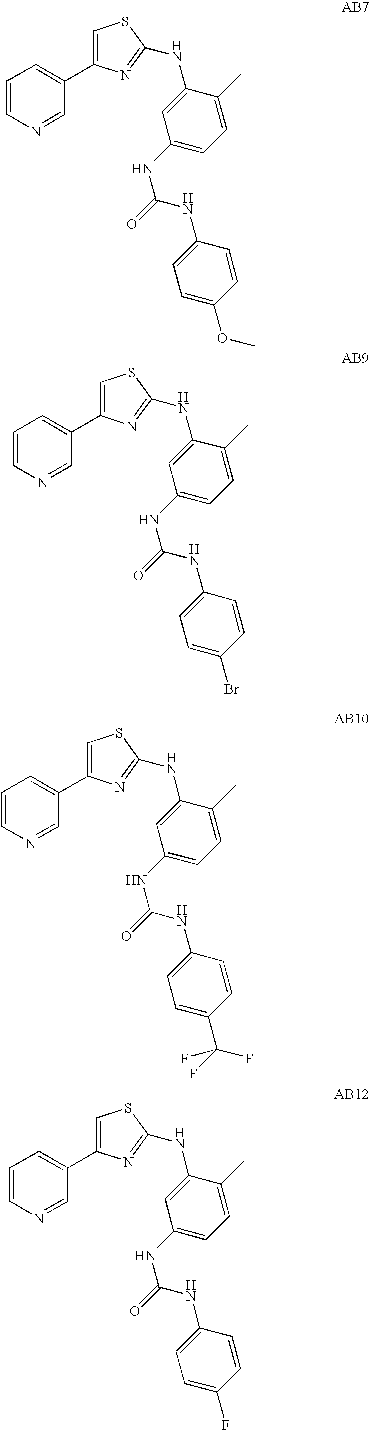

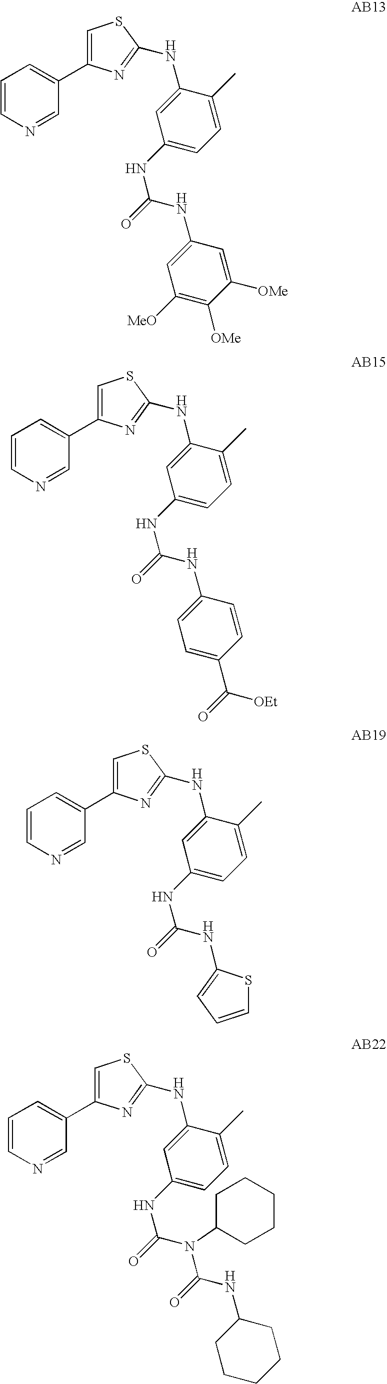

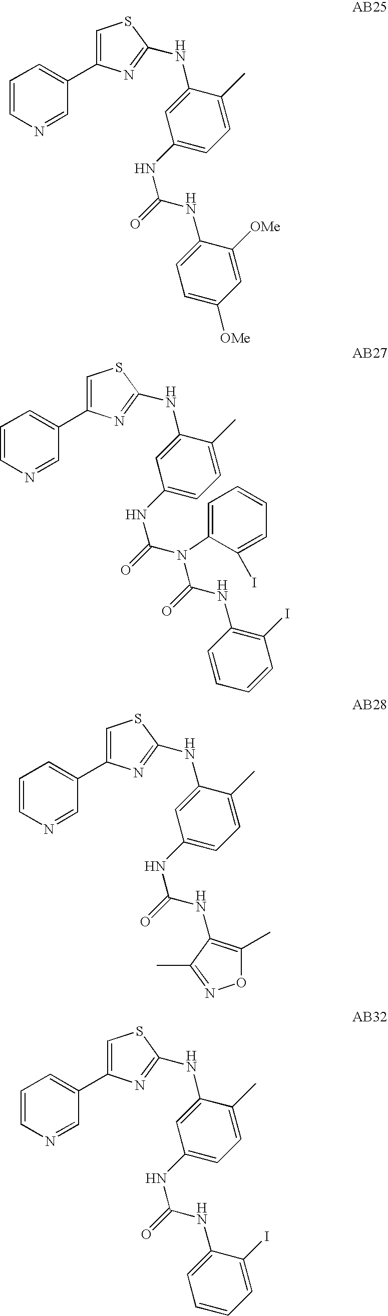

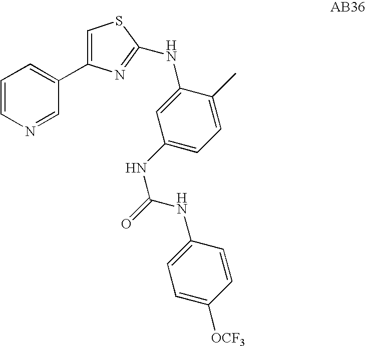

Among the particular compounds in which R1 has the meaning as depicted in d) above, the invention is directed to compounds of the following formula VIIbis:

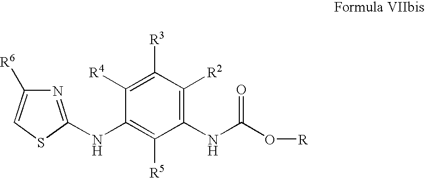

Wherein R, and R2, R3, R4, R5 and R6 are as defined above.

For example, the invention is aimed at:

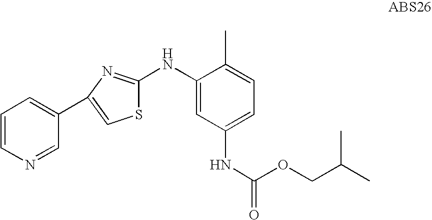

Among the compounds of formula III, the invention is particularly embodied by the compounds of the following formula VIII:

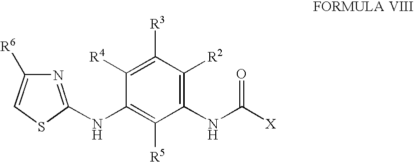

wherein X is R or NRR′ and wherein R and R′ are independently chosen from H, an aryl, an heteroaryl, an alkyl and a cycloalkyl group optionally substituted with at least one heteroatom, such as for example a halogen chosen from F, I, Cl and Br and optionally bearing a pendant basic nitrogen functionality; or an aryl, an heteroaryl, an alkyl and a cycloalkyl group substituted with an aryl, an heteroaryl, an alkyl and a cycloalkyl group optionally substituted with at least one heteroatom, such as for example a halogen chosen from F, I, Cl and Br and optionally bearing a pendant basic nitrogen functionality,

R² is hydrogen, halogen or a linear or branched alkyl group containing from 1 to 10 carbon atoms, trifluoromethyl or alkoxy;

R³ is hydrogen, halogen or a linear or branched alkyl group containing from 1 to 10 carbon atoms, trifluoromethyl or alkoxy,

R⁴ is hydrogen, halogen or a linear or branched alkyl group containing from 1 to 10 carbon atoms, trifluoromethyl or alkoxy;

R⁵ is hydrogen, halogen or a linear or branched alkyl group containing from 1 to 10 carbon atoms, trifluoromethyl or alkoxy;

R⁶ is one of the following:

(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;

(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;

(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy. It will be understood that the NH—CO—X bound to the phenyl group can also be a CO—NH—X group.

Among the preferred compounds corresponding formula VIII, the invention is directed to compounds in which X is a substituted alkyl, aryl or heteroaryl group bearing a pendant basic nitrogen functionality represented for example by the structures a to f shown below, wherein the wavy line corresponds to the point of attachment to core structure of formula VIII:

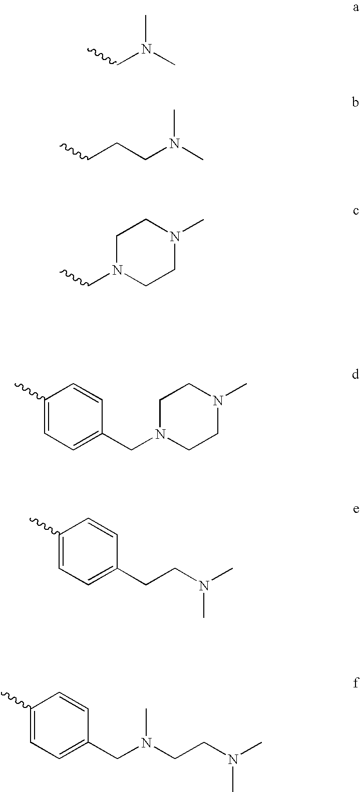

Among group a to f, R¹ is preferentially group d.

Furthermore, among the preferred compounds of formula IV or XI, the invention concerns the compounds in which R² and R³ are hydrogen. Preferentially, R⁴ is a methyl group and R⁵ is H. In addition, R⁶ is preferentially a 3-pyridyl group (cf structure g below) wherein the wavy line corresponds to the point of attachment to core structure of formula III or VIII.

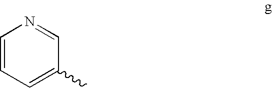

More specifically, the invention relates to the above method which further 20 comprises the combined, sequential, simultaneous or separate administration of calcitriol or analogs thereof and a c-kit inhibitor selected from compounds of formula IX as depicted above, wherein:
X is group d and R⁶ is a 3-pyridyl group,
X is group d and R⁴ is a methyl group,
R¹ is group d and R² is H,
R¹ is group d and R³ is H,
R¹ is group d and R² and/or R³ and/or R⁵ is H,
R⁶ is a 3-pyridyl group and R³ is a methyl group,
R⁶ is a 3-pyridyl group and R² is H,
R² and/or R³ and/or R⁵ is H and R⁴ is a methyl group,
R² and/or R³ and/or R⁵ is H, R⁴ is a methyl group and R⁶ is a 3-pyridyl group.

One compound of formula VIII can be:

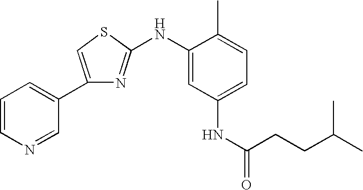

ABS27

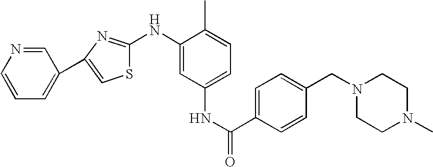

AB60 5(2-(2-methyl-5-amino)phenyl-4-(3-pyridyl)-thiazole)

In the above Formula III, IV, V, VI, VII, VIII, and IX, the invention also contemplates the compounds wherein one cetone group is added to the thiazole core. In addition, when reference is made to a C1-C10 alkyl group, it will be understood that it embraces C1-C3, C2-C4, C2-C5, C3-C6, C2 or C3-C10.

In addition, the invention is directed to the use of compounds capable of specifically inhibiting c-kit wild or both c-kit wild and/or juxtamembrane mutant c-kit to manufacture a medicament for treating the category of patients that are not bearing the 816 mutation and are afflicted with Category I, II, III and IV mastocytosis, in particular mastocytosis with an associated hematological disorder, such as a myeloproliferative or myelodysplastic syndrome, or acute leukemia, myeloproliferative disorder associated with mastocytosis, and mast cell leukemia.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

For treating category II, III and IV mastocytosis, oral, intravenous and intramuscular route of administration are preferred.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly concentrated solutions.

The pharmaceutical composition may be provided as a salt and can be formed with many acids, including but not limited to, hydrochloric, sulfuric, acetic, lactic, tartaric, malic, and succine, acids, etc. Salts tend to be more soluble in aqueous or other protonic solvents than are the corresponding free base forms. In other cases, the preferred preparation may be a lyophilized powder which may contain any or all of the following: 1-50 mM histidine, 0. 1%-2% sucrose, and 2-7% mannitol, at a pH range of 4.5 to 5.5, that is combined with buffer prior to use.

Pharmaceutical compositions suitable for use in the invention include compositions wherein c-kit inhibitors are contained in an effective amount to achieve the intended purpose. The determination of an effective dose is well within the capability of those skilled in the art. A therapeutically effective dose refers to that amount of active ingredient, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions which exhibit large therapeutic indices are preferred.

In a still further embodiment, the invention is directed to a composition comprising a c-kit inhibitors for topical application. Such composition is adapted for treating skin disorders such as solitary mastocytoma and bullous, erythrodermic and teleangiectatic mastocytosis.

The compositions according to the invention may be presented in all forms normally used for topical application, in particular in the form of a gel, paste, ointment, cream, lotion, liquid suspension aqueous, aqueous-alcoholic or, oily solutions, or dispersions of the lotion or serum type, or anhydrous or lipophilic gels, or emulsions of liquid or semi-solid consistency of the milk type, obtained by dispersing a fatty phase in an aqueous phase or vice versa, or of suspensions or emulsions of soft, semi-solid consistency of the cream or gel type, or alternatively of microemulsions, of microcapsules, of microparticles or of vesicular dispersions to the ionic and/or nonionic type. These compositions are prepared according to standard methods.

The composition according to the invention comprises any ingredient commonly used in dermatology and cosmetic. It may comprise at least one ingredient selected from hydrophilic or lipophilic gelling agents, hydrophilic or lipophilic active agents, preservatives, emollients, viscosity enhancing polymers, humectants, surfactants, preservatives, antioxidants, solvents, and fillers, antioxidants, solvents, perfumes, fillers, screening agents, bactericides, odor absorbers and coloring matter.

As oils which can be used in the invention, mineral oils (liquid paraffin), vegetable oils (liquid fraction of shea butter, sunflower oil), animal oils, synthetic oils, silicone oils (cyclomethicone) and fluorinated oils may be mentioned. Fatty alcohols, fatty acids (stearic acid) and waxes paraffin, camauba, beeswax) may also be used as fatty substances.

As emulsifiers which can be used in the invention, glycerol stearate, polysorbate 60 and the PEG-6/PEG-32/glycol stearate mixture are contemplated.

As hydrophilic gelling agents, carboxyvinyl polymers (carbomer), acrylic copolymers such as acrylate/alkylacrylate copolymers, polyacrylamides, polysaccharides such as hydroxypropylcellulose, clays and natural gums may be mentioned, and as lipophilic gelling agents, modified clays such as bentones, metal salts of fatty acids such as aluminum stearates and hydrophobic silica, or alternatively ethylcellulose and polyethylene may be mentioned.

As hydrophilic active agents, proteins or protein hydrolysates, amino acids, polyols, urea, allantoin, sugars and sugar derivatives, vitamins, starch and plant extracts, in particular those of Aloe vera may be used.

As lipophilic active, agents, retinol (vitamin A) and its derivatives, tocopherol (vitamin E) and its derivatives, essential fatty acids, ceramides and essential oils may be used. These agents add extra moisturizing or skin softening features when utilized.

In addition, a surfactant can be included in the composition so as to provide deeper penetration of the ingredients of the c-kit inhibitor.

Among the contemplated ingredients, the invention embraces penetration enhancing agents selected for example from the group consisting of mineral oil, water, ethanol, triacetin, glycerin and propylene glycol; cohesion agents selected for example from the group consisting of polyisobutylene, polyvinyl acetate and polyvinyl alcohol, and thickening agents.

Chemical methods of enhancing topical absorption of drugs are well known in the art. For example, compounds with penetration enhancing properties include sodium lauryl sulfate (Dugard, P. H. and Sheuplein, R. J., “Effects of Ionic Surfactants on the Penneability of Human Epidermis: An Electrometric Study,” J. Ivest. Dermatol., V.60, pp. 263-69, 1973), lauryl amine oxide (Johnson et. al., U.S. Pat. No. 4,411,893), azone (Rajadhyaksha, U.S. Pat. Nos. 4,405,616 and 3,989,816) and decylmethyl sulfoxide (Sekura, D. L. and Scala, J., “The Percutaneous Absorption of Alkylmethyl Sulfides,” Pharmacology of the Skin, Advances In Biolocy of Skin, (Appleton-Century Craft) V.12, pp. 257-69, 1972). It has been observed that increasing the polarity of the head group in amphoteric molecules increases their penetration-enhancing properties but at the expense of increasing their skin irritating properties (Cooper, E. R. and Bemer, B., “Interaction of Surfactants with Epidermal Tissues: Physiochemical Aspects,” Surfactant Science Series, V. 16, Reiger, M. M. ed. (Marcel Dekker, Inc.) pp. 195-210, 1987).

A second class of chemical enhancers are generally referred to as co-solvents. These materials are absorbed topically relatively easily, and, by a variety of mechanisms, achieve permeation enhancement for some drugs. Ethanol (Gale et. al., U.S. Pat. No. 4,615,699 and Campbell et. al., U.S. Pat. Nos. 4,460,372 and 4,379,454), dimethyl sulfoxide (U.S. Pat. Nos. 3,740,420 and 3,743,727, and U.S. Pat. No. 4,575,515), and glycerine derivatives (U.S. Pat. No. 4,322,433) are a few examples of compounds which have shown an ability to enhance the absorption of various compounds.

The invention is also directed to a method for treating category IV mastocytosis including mast cell leukemia, comprising administering a c-kit inhibitors as defined above depending on the presence or absence of the 816 mutation in c-kit and a compound selected from 2-Chloro-2′-desoxyadenosine and analogs thereof. 2-Chloro-2′-desoxyadenosine (2-CDA), Cladribine, Merck Index (12th ed.) # 2397 has the following formula:

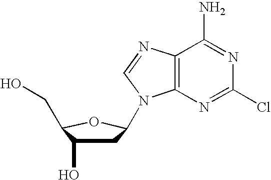

In another embodiment, the invention relates to a method for diagnosing an individual suspected to be afflicted with category I, II, III and IV mastocytosis or symptoms associated with mast cells proliferation comprising a) extracting RNA from a skin sample, b) Reverse transcription of the RNA using oligo dT primers and random primers, c) amplifying the cDNA obtained in step b) using primers:

| | |
|---|---|
| U2 | |
| (5′ GGATGACGAGTTGGCCCTAGA 3′) | (SEQ ID No 1) |
| and | |
| L1 | |
| (5′ GTAGAACTTAGAATCGACCGGCA 3′), | (SEQ ID No 2) |

and detecting the presence or the absence of a mutation of the c-kit c-DNA at position 816 of C-KIT.

In other words, it is aimed at a method comprising the use of SEQ ID No 1 and SEQ ID No 2 for amplifying and detecting specifically the presence or absence of the cDNA corresponding to the ‘816’ mutation of C-KIT from cDNAs obtained from reversed transcribed skin total RNA sample. The following primers can also be used:

| | |
|---|---|
| (sens) | |
| 5′ ATCCTCCTTACTCATGGTCGGATC 3′ | (SEQ ID No 5) |
| (antisens) | |
| 5′ CGACCGGCATTCCAGGATAG 3′ | (SEQ ID No 6) |

It will be understood that the invention is aimed at these primers as well as any primers displaying non significant differences in the sequence frame or length.

Utility of the invention will further ensue from the detailed description below.

### EXAMPLE 1

### Mutation Assessment in Patients

### Material and Method

### RNA Extractions

A section of frozen skin at −80° C. is poured in 500 μl RLT media (+5 μl of βmercaptoéthanol), which correspond to the buffer of the QIAGEN Rneasy Mini kit. It is grinded in a T25 basic ultra-turrax homogenizer and an ultra-turrax axis of 0.8 cm (ref B35333 de Fisher Bioblock Scientific). The grinded skin section is then frozen before RNA extraction.

Before each grinding, the ultra-turrax rod is rinsed twice in two 50 ml tubes containing non autoclaved and non DEPC treated mili Q water. The rod is then rinsed and cleaned with 250 ml ethanol 70% during 5 minutes. Elle est ensuite déposée dans un incubateur à 37° C. pendant 45 minutes (étape de séchage) et remontée juste avant l'extraction suivante. Further steps are necessary to ensure no RNA degradation during grinding.

For the following tests a negative control is used. Two skin sections are extracted successively from the patient and are checked for the presence of the valine mutation at position 816 of the KIT receptor.

RNA is extracted from the grinded skin tumor with the QIAGEN (Rneasy Mini) kit. Each sample is thawed. The extraction is performed with a minimum of 4 samples to minimize risks of RNA degradation. 983 μl of milli Q water is added to 500 μl of RLT buffer and 17 μl of proteinase K (ref 19133 QIAGEN). We incubate 10 min at 55° C. and centrifuge 3 min at 10 000 g at room temperature. The supernatant is mixed with 750 ml absolute ethanol. 700 μl of supernatant is run on a Rneasy minispin column and centrifuged at room temperature during 15 s at 8000 g. The remaining supernatant is run in the same column. We clean with 350 μl RW1 buffer. After centrifugation at 8000 q during 15 s, 10 μl of DNAse and 70 μl of RDD buffer is poured in the column (RNAse free DNAse set : ref 79254 de QIAGEN SA).

DNAse reacts during 15 minutes at room temperature. Then, 350 μl of RW1 buffer are added on the column to rinse (centrifigation of 15 sec at 8000 g).

The column is rinsed with RPE buffer then centrifuged 2 minutes à 8000 g to eliminate the RPE buffer. The RNA is then washed out elué with 30 μl of DEPC treated water and kept frozen at −80° C.

Reverse Transcriptase Step 20 ng of RNA are used for the synthesis of the CDNA. We use the ProStar™ first strand RT-PCR kit of Stratagene. Then final volume of the reverse transcriptase reaction is 25 μl.

In a final volume of 17.5 μl containing 200 ng of RNA, we add 1.5 μl of oligo dT and 1.5 μl of random primers. The reaction mixture is incubated 5 min at 65° C. then 10 minutes at room temperature. The synthesis of the first strand is performed after adding 2.5 μl of =first strand 10X buffer, 0.5 μl RNAse block ribonuclease inhibitor, 1 μl of dNTP 100 mM, 0.5 μl of reverse transcriptase StrataScript™ and it is incubated during 1 hour at 42° C.

### Amplification of the c-kit Gene

2.5 μl of the reverse transcriptase reaction is used for amplification. The amplification program is as follows:

| | |
|---|---|
| 94° C. 10 min | |
| 94° C. 15 sec | |
| 55° C. 15 sec | 35 cycles |
| 72° C. 30 sec | |
| 72° C. 7 min | |
| 4° C. 15 h | |

Primers are U2 et L1:

| | |
|---|---|
| U2: | |
| 5′ GGATGACGAGTTGGCCCTAGA 3′ | (SEQ ID No 1) |
| L1: | |
| 5′ GTAGAACTTAGAATCGACCGGCA 3′ | (SEQ ID No 2) |

In case the quantity of amplified cDNA is not enough for sequencing, a nested-PCR is used using the above program and the following primers:

| | |
|---|---|
| U2217: | |
| 5′ CCCAACCAAGGCCGACAAAAGGA 3′ | (SEQ ID No 3) |
| L2722: | |
| 5′ TCCCAGCAAGTCTTCATTATGTC 3′ | (SEQ ID No 4) |

### Purification of PCR products

This step is performed using the GENECLEAN III® kit.

### Sequencing

U2 and L1 primers are used for the sequencing reaction. The following primers can also be used:

| | |
|---|---|
| (sens) | |
| 5′ ATCCTCCTTACTCATGGTCGGATC 3′ | (SEQ ID No 5) |
| (antisens) | |
| 5′ CGACCGGCATTCCAGGATAG 3′ | (SEQ ID No 6) |

### EXAMPLE 2

### Human c-kit 816 Harboring Mast Cells Are Resistant to the Antiproliferative Activity of STI-571

The following experiment has been performed in order to confirm that mast cells harboring the human kit 816 mutant are resistant to the antiproliferative activity of STI-571, an inhibitor of kit wild-type activity.

For this purpose, a mouse bone marrow-derived mast cell line (BMMC), factor-independent and expressing the mutant D816V form of the human c-kit (as assessed by RT-PCR with specific primers for the mutant form of the human c-kit) has been generated by long-term liquid culture of bone marrow-derived progenitors from mice transgenic for the mutant form of the human c-kit. This cell line (doubling time of around 48 hours) has then been treated with increasing concentrations of STI-571 (0 to 10⁻⁵ M) for 3 days. At 24, 48 and 72 hours, the number of viable cells was determined in each condition using the trypan blue exclusion assay. The proliferation of the BMMC line with the mutant D816V form of the human c-kit was not affected, at any time, by STI-571 under or equal to 1 microM (FIG. 2). By contrast, the proliferation of these BMMC was dose- and time-dependently inhibited by concentrations of STI-571 above 1 microM. At this concentration, it is not possible to use STI-571 for treating patients afflicted with proliferative mast expressing the ‘816’ mutant C-KIT. This result is also observed using the lymphoid BaF/3 murine cell line transfected with the D816V mutant form of the human c-kit.

In conclusion, the D816V mutant form of the human c-kit receptor is resistant to pharmacological doses of STI 571.

We then choose to screen other compounds for specifically treating patients afflicted with proliferative mast expressing the ‘816’ mutant C-KIT. In this regards, we have shown that compounds of formula III and

1. A method for a tailored treatment of mastocytosis comprising
a) assessing whether or not a c-kit mutation at position 816 is detected in a sample of a patient in need of the treatment; and
b) administering to the patient 816 c-kit mutant inhibitor in case the mutation is detected or an inhibitor displaying efficacy on c-kit wild and/or on juxtamembrane mutated c-kit in case the mutation is not detected.

2-9. (canceled)

10. The method of claim 1, wherein the mastocytosis is category I mastocytosis, category II mastocytosis, category III mastocytosis, category IV mastocytosis or a symptom associated thereof.

11. The method of claim 1, wherein the mastocytosis is urticaria pigmentosa, diffuse cutaneous mastocytosis, solitary mastocytoma in human, dog mastocytoma, bullous mastocytosis, erythrodermic mastcytosis, teleangiectatic mastocytosis, mastocytosis with an associated hematological disorder, myeloproliferative disorder associated with mastocytosis or mast cell leukemia.

12. The method of claim 1, wherein the mastocytosis is category IV mastocytosis and wherein the method further comprises administering to the patient a compound selected from a group consisting of 2-Chloro-2′-desoxyadenosine and analogs thereof.

13. A method of treating Category II, III or IV mastocytosis in a patient having a c-kit mutation at position 816, said method comprising
administering to the patient a 816 c-kit mutant inhibitor.

14. The method of claim 13, wherein the 816 c-kit mutant inhibitor is a compound of formula III 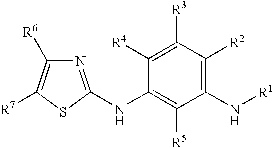
wherein R¹ is
a) a linear or branched alkyl group containing from 1 to 10 carbon atoms optionally substituted with a least one heteroatom or bearing a pendant basic nitrogen functionality;
b) an aryl or heteroaryl group optionally substituted with alkyl or aryl group, optionally substituted with at least one heteroatom or bearing a pendant basic nitrogen functionality;
c) a sulfonyl or a —SO₂R group wherein R is an alkyl, aryl or heteroaryl substituted with a heteroatom or bearing a pendant basic nitrogen functionality; or
d) a —CO—NH—R, —CO—R, —CO—OR or a —CO—NRR′, wherein R and R′ are independently selected from a group consisting of H, aryl group optionally substituted with at least one heteroatom or bearing a pendant basic nitrogen functionality, heteroaryl optionally substituted with at least one heteroatom or bearing a pendant basic nitrogen functionality, alkyl optionally substituted with at least one heteroatom or bearing a pendant basic nitrogen functionality, cycloalkyl optionally substituted with at least one heteroatom or bearing a pendant basic nitrogen functionality;
R² is hydrogen, halogen, a linear or branched alkyl group containing from 1 to 10 carbon atoms, trifluoromethyl or alkoxy;
R³ is hydrogen, halogen, a linear or branched alkyl group containing from 1 to 10 carbon atoms, trifluoromethyl or alkoxy;
R⁴ is hydrogen, halogen, a linear or branched alkyl group containing from 1 to 10 carbon atoms, trifluoromethyl or alkoxy;
R⁵ is hydrogen, halogen, a linear or branched alkyl group containing from 1 to 10 carbon atoms, trifluoromethyl or alkoxy;
R⁶ is
(i) an aryl group optionally substituted with one or substituents selected from the group consisting of halogen, a linear or branched alkyl group containing from 1 to 10 carbon atoms, trifluoromethyl and alkoxy;
(ii) a heteroaryl group optionally substituted with one or more substituents selected from the group consisting of halogen, a linear or branched alkyl group containing from 1 to 10 carbon atoms, trifluoromethyl and alkoxy;
(iii) a five-member aromatic heterocyclic group optionally substituted with one or substituents selected from the group consisting of halogen, a linear or branched alkyl group containing from 1 to 10 carbon atoms, trifluoromethyl and alkoxy; or
(iv) H, I, F, Br, Cl, NH₂, NO₂ or SO₂;
and R⁷ is
(i) an aryl group optionally substituted with one or substituents selected from the group consisting of halogen, a linear or branched alkyl group containing from 1 to 10 carbon atoms, trifluoromethyl and alkoxy;
(ii) a heteroaryl group optionally substituted with one or more substituents selected from the group consisting of halogen, a linear or branched alkyl group containing from 1 to 10 carbon atoms, trifluoromethyl and alkoxy;
(iii) a five-member aromatic heterocyclic group optionally substituted with one or substituents selected from the group consisting of halogen, a linear or branched alkyl group containing from 1 to 10 carbon atoms, trifluoromethyl and alkoxy; or
(iv) H, I, F, Br, Cl, NH₂, NO₂ or SO₂.

15. A method of treating Category I, II, III or IV mastocytosis in a patient not bearing a c-kit mutation at position 816, said method comprising
administering to the patient an inhibitor displaying efficacy on c-kit wild and/or on juxtamembrane mutated c-kit.

16. The method of claim 15, wherein the inhibitor is a compound of formula III: 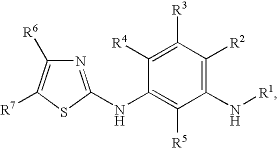
wherein R¹ is
a) a linear or branched alkyl group containing from 1 to 10 carbon atoms optionally substituted with a least one heteroatom or bearing a pendant basic nitrogen functionality;
b) an aryl or heteroaryl group optionally substituted with alkyl or aryl group, optionally substituted with at least one heteroatom or bearing a pendant basic nitrogen functionality;
c) a sulfonyl or a —SO₂R group wherein R is an alkyl, aryl or heteroaryl substituted with a heteroatom or bearing a pendant basic nitrogen functionality; or
d) a —CO—NH—R, —CO—R, —CO—OR or a —CO—NRR′, wherein R and R¹ are independently selected from a group consisting of H, aryl group optionally substituted with at least one heteroatom or bearing a pendant basic nitrogen functionality, heteroaryl optionally substituted with at least one heteroatom or bearing a pendant basic nitrogen functionality, alkyl optionally substituted with at least one heteroatom or bearing a pendant basic nitrogen functionality, cycloalkyl optionally substituted with at least one heteroatom or bearing a pendant basic nitrogen functionality;
R² is hydrogen, halogen, a linear or branched alkyl group containing from 1 to 10 carbon atoms, trifluoromethyl or alkoxy;
R³ is hydrogen, halogen, a linear or branched alkyl group containing from 1 to 10 carbon atoms, trifluoromethyl or alkoxy;
R⁴ is hydrogen, halogen, a linear or branched alkyl group containing from 1 to 10 carbon atoms, trifluoromethyl or alkoxy;
R⁵ is hydrogen, halogen, a linear or branched alkyl group containing from 1 to 10 carbon atoms, trifluoromethyl or alkoxy;
R⁶ is
(i) an aryl group optionally substituted with one or substituents selected from the group consisting of halogen, a linear or branched alkyl group containing from 1 to 10 carbon atoms, trifluoromethyl and alkoxy;
(ii) a heteroaryl group optionally substituted with one or more substituents selected from the group consisting of halogen, a linear or branched alkyl group containing from 1 to 10 carbon atoms, trifluoromethyl and alkoxy;
(iii) a five-member aromatic heterocyclic group optionally substituted with one or substituents selected from the group consisting of halogen, a linear or branched alkyl group containing from 1 to 10 carbon atoms, trifluoromethyl and alkoxy; or
(iv) H, I, F, Br, Cl, NH₂, NO₂ or SO₂;
and R⁷ is
(i) an aryl group optionally substituted with one or substituents selected from the group consisting of halogen, a linear or branched alkyl group containing from 1 to 10 carbon atoms, trifluoromethyl and alkoxy;
(ii) a heteroaryl group optionally substituted with one or more substituents selected from the group consisting of halogen, a linear or branched alkyl group containing from 1 to 10 carbon atoms, trifluoromethyl and alkoxy;
(iii) a five-member aromatic heterocyclic group optionally substituted with one or substituents selected from the group consisting of halogen, a linear or branched alkyl group containing from 1 to 10 carbon atoms, trifluoromethyl and alkoxy; or
(iv) H, I, F, Br, Cl, NH₂, NO₂ or SO₂.

17. The method of claim 15, wherein the inhibitor is a N-phenyl-2-pyrimidine-amine compound of formula II 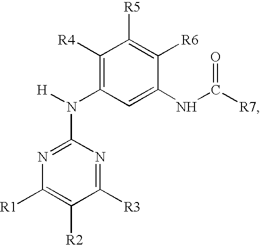
wherein R1, R2 and R3 are each independently selected from the group consisting of H, F, Cl, Br, I, C1-C5 alkyl group, cyclic group and heterocyclic group,
wherein R4, R5 and R6 are each independently selected from H, F, Cl, Br, I, and C1-C5 alkyl group,
and wherein R7 is phenyl group substituted with at least one substituent, said substituent has at least one basic site.

18. A method of diagnosing mastocytosis in an individual, said method comprising
a) reversing transcription of a RNA sample from a skin of the individual using oligo dT primers and random primers to obtain a cDNA;
b) amplifying the cDNA using primers

| | |
|---|---|
| U2 | |
| (5′ GGATGACGAGTTGGCCCTAGA 3′) | (SEQ ID NO 1) |
| and | |
| L1 | |
| (5′ GTAGAACTTAGAATCGACCGGCA 3′), | (SEQ ID NO 2) |
| or | |
| 5′ ATCCTCCTTACTCATGGTCGGATC 3′ | (SEQ ID NO 5) |
| 5′ CGACCGGCATTCCAGGATAG 3′ | (SEQ ID NO 6) |

c) detecting the presence or the absence of the c-kit of c-DNA corresponding to 816 position of the c-kit.


SEQUENCE LISTING (6 sequences)

SEQ ID NO: 1 - DNA, 21 bp, Artificial
  U2 Primer
ggatgacgagttggccctaga

SEQ ID NO: 2 - DNA, 23 bp, Artificial
  L1 Primer
gtagaacttagaatcgaccggca

SEQ ID NO: 3 - DNA, 23 bp, Artificial
  U2217 Primer
cccaaccaaggccgacaaaagga

SEQ ID NO: 4 - DNA, 23 bp, Artificial
  L2722 Primer
tcccagcaagtcttcattatgtc

SEQ ID NO: 5 - DNA, 24 bp, Artificial
  Sense Primer
atcctccttactcatggtcggatc

SEQ ID NO: 6 - DNA, 20 bp, Artificial
  Antisense Primer
cgaccggcattccaggatag